(19) United States
(12) Patent Application Publication
(10) Pub. No.: US 2017/0058025 A1
(43) Pub. Date: Mar. 2, 2017

(54) METHODS OF USING ANTI-ANG2 ANTIBODIES

(71) Applicant: Medlmmune, LLC, Gaithersburg, MD (US)

(72) Inventors: Dominic LAI, Daly City, CA (US); Robert SIKORSKI, Woodside, CA (US); David HYMAN, New York, NY (US); Naiyer A. RIZVI, New York, NY (US)

(21) Appl. No.: 15/308,187

(22) PCT Filed: May 6, 2015
(86) PCT No.: PCT/US2015/029447

§ 371 Date: Nov. 1, 2016

Related U.S. Application Data
(60) Provisional application No. 62/005,525, filed on May 30, 2014.
(60) Provisional application No. 61/989,884, filed on May 7, 2014.

(51) Int. Cl. C07K 16/22; A61K 31/337; A61K 31/555; A61K 39/395; A61K 31/517

(52) U.S. Cl. CPC C07K 16/22; A61K 39/39558; A61K 31/517; A61K 31/555; A61K 31/337; C07K 2317/565; A61K 2039/507

(57) ABSTRACT

This application provides methods of treating cancer and/or inhibiting angiogenesis with an anti-Ang2 antibody or functional part thereof either alone or in combination with at least one additional therapeutic agent.

### FIELD

This application relates to the field of biotechnology and medicine.

### BACKGROUND

MEDI1/5 is a human IgG1κ antibody which preferentially binds to angiopoietin 2 (Ang2), and to a much lesser extent, Ang1. Ang2 is a proangiogenic cytokine which exhibits broad expression in the remodeling vasculature of human tumors, but limited expression in normal tissues, making it an attractive candidate target for antiangiogenic cancer therapy. Growing evidence supports the hypothesis that blocking Ang2-Tie2 receptor interactions would be an effective antiangiogenic therapy for the treatment of solid tumors.

Ang2 is almost exclusively expressed by endothelial cells. Ang2 upregulation has been observed in response to stress, such as hypoxia, as well as cytokine and angiogenic stimulation by histamine, VEGF, and FGF. In normal adult tissue, Ang2 is detectable in ovary, placenta and uterus, which are predominant sites of vascular remodeling. In neoplastic settings, increased Ang2 expression has been correlated spatially with areas of angiogenesis (e.g., breast, colon, lung, renal, prostate, and ovarian cancers). Increased expression of Ang2 shifts the balance of vessel growth to a more plastic state that is responsive to additional proangiogenic cytokines such as VEGF, as well as recruitment of Tie2-expressing monocytes (TEMs) to tumors. Much like VEGF, elevated Ang2 expression has been identified in renal, colon, lung, breast, liver, prostate, gastric, ovarian and melanoma skin cancers, as well as in gliomas. Furthermore, increased Ang2 expression has been correlated with worse histological grade, more advanced tumor stage, and adverse prognosis in colorectal, gastric, breast and bladder cancers, as well as glioblastoma multiforme (GBM). In non-small cell lung cancer (NSCLC), higher Ang2 expression has also been correlated with poorer overall survival. Elevated expression of Ang2 at sites of vascular remodeling in tumors coupled with its limited role in normal tissues makes it an excellent target for antiangiogenic cancer therapy.

While a few antiangiogenic drugs have shown significant clinical activity as monotherapy, clinical experience suggests that antiangiogenic therapies are likely to be more effective when co-administered with other therapeutic interventions. Bevacizumab was first approved in 2004 in combination with intravenous 5-fluorouracil-based chemotherapy in the first-line setting for patients with metastatic carcinoma of the colon or rectum. It was later approved in conjunction with second-line treatment of colorectal cancer (CRC) as well as other solid tumors including nonsquamous non-small cell lung cancer (NSCLC) (with carboplatin and paclitaxel, first-line treatment), glioblastoma (single agent for recurrent disease) and metastatic renal cell carcinoma (mRCC) (with interferon alpha).

Thus, there exists a need for developing appropriate treatment regimens for anti-Ang2 antibody therapy, either alone or in combination with other agents.

### SUMMARY

In accordance with the description, disclosed is a method of treating cancer or inhibiting angiogenesis in a patient comprising
a. providing an anti-Ang2 antibody or functional part thereof,
b. administering an anti-Ang2 antibody or functional part thereof to the patient, wherein the anti-Ang2 antibody or functional part thereof is administered at a dose from about 200 mg to about 1500 mg.

In another aspect, the antibody or functional part thereof comprises the same heavy and light chain CDRs as MEDI1/5.

In one embodiment, the antibody or functional part thereof is MEDI1/5 or a functional part thereof.

In one mode, the anti-Ang2 antibody or functional part thereof is administered at a dose from about 200 mg to about 1000 mg.

In a further aspect, the anti-Ang2 antibody or functional part thereof is administered at a dose from about 300 mg to about 1500 mg.

In yet another embodiment, the anti-Ang2 antibody or functional part thereof is administered at a dose from about 1000 mg to about 1500 mg.

In a further iteration, the anti-Ang2 antibody or functional part thereof is administered at a dose of about 1000 mg.

In one embodiment, the anti-Ang2 antibody or functional part thereof is administered at a dose of about 1500 mg.

In a further mode, the anti-Ang2 antibody or functional part thereof is administered an IV infusion over from about 60 to about 90 minutes.

In one aspect, the patient receives multiple doses.

In another aspect, the dosage cycle is about every 14 days

In one embodiment, the dosage cycle is about every 21 days.

In a further embodiment, the anti-Ang2 is coadministered with at least one additional therapeutic agent.

In a further mode, at least one additional therapeutic agent is chosen from at least one of carboplatin, capecitabine, gemcitabine, or paclitaxel.

In an additional aspect, at least one additional therapeutic agent is carboplatin and paclitaxel.

In a further mode, at least one additional therapeutic agent is cediranib.

In a further embodiment, at least one additional therapeutic agent is an anti-VEGF antibody or functional part thereof.

In another mode, the antibody is bevacizumab.

In an additional aspect, the patient has ovarian cancer.

In another mode, the patient has glioblastoma multiforme.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides the overall clinical study design.

FIG. 2A illustrates the mean serum MEDI1/5 concentration-time profiles for MEDI1/5 administered at 5, 10, 20, 100, 300, 1000, and 1500 mg. Mean serum concentrations increased with an increase of MEDI1/5 dose levels.

FIG. 2B shows the mean serum MEDI1/5 concentration-time profiles for MEDI1/5 administered at 60 mg Q2W, 200 mg Q2W, 600 mg Q23W, and 1000 mg Q2W. Mean serum concentrations increased with an increase of MEDI1/5 dose levels.

FIG. 3 illustrates total Ang2 levels concentration-time profiles for MEDI1/5 administered at 5, 10, 20, 100, 300, 1000, and 1500 mg.

FIG. 4 provides results of brain scans in a patient diagnosed with gliosarcoma.

FIG. 5 provides results of brain scans in a patient diagnosed with glioblastoma multiforme.

FIG. 6 provides additional scans from the patient diagnosed with glioblastoma multiforme from FIG. 5.

FIG. 7 provides additional scans from the patient diagnosed with glioblastoma multiform from FIGS. 5 and 6.

### DESCRIPTION OF THE SEQUENCES

Table 1 provides a listing of certain sequences referenced in present embodiments. The CDRs are provided in bold.

TABLE 1

| | | SEQ |
|---|---|---|
| | | ID |
| Description | Sequence | NO |
| MEDI1/5 heavy | QVQLVESGGGVVQPGRSLRLSCAASGFTFTN | 1 |
| chain variable | YGMHWVRQAPGKGLEWVAVISHDGNNKYYVD | |
| region | SVKGRFTISRDNSKNTLYLQMNSLRAEDTAV | |
| | YYCAREGIDFWSGLNWFDPWGQGTLVTVSS | |
| MEDI1/5 light | EIVLIQSPGILSLSPGERATLSCRASQSITG | 2 |
| chain variable | SYLAWYQQKPGQAPRLLITGASSWATGIPDR | |
| region | FSGSGSGTDFTLTISRLEPEDFAVYYCQQYS | |
| | SSPITFGQGTRLEIK | |

### DESCRIPTION OF THE EMBODIMENTS

### I. Methods of Treatment

A. Methods of Treating Cancer

One embodiment encompasses a method of treating cancer in a patient comprising
a. providing an anti-Ang2 antibody or functional part thereof,
b. administering an anti-Ang2 antibody or functional part thereof to the patient, wherein the anti-Ang2 antibody or functional part thereof is administered at a dose from about 300 mg to about 1500 mg.

High levels of both vascular endothelial growth factor (VEGF) and Ang2 present in breast cancer, NSCLC, ovarian cancer, and acute myeloid leukemia have been shown to correlate with a worse prognosis than those tumor types with either VEGF or Ang2 elevation alone. It is hypothesized that targeting Ang2, which has been reported to be involved not only in angiogenesis, but also in metastasis and inflammation, may enhance the efficacy of anti-VEGF treatments. One role of Ang2 in angiogenesis appears to be in cooperation with VEGF-A function. Ang2 destabilizes vasculature and initiates angiogenesis in the presence of proangiogenic factors such as VEGF or in the absence of VEGF-A. Ang2 also induces apoptosis of endothelial cells and blood vessel regression. Therefore, in one embodiment, an anti-Ang2 antibody or functional part thereof (such as MEDI1/5) may be combined with bevacizumab, an anti-VEGF antibody, to improve control of angiogenesis in solid tumors. In another embodiment, an anti-Ang2 antibody or functional part thereof (such as MEDI1/5) may be provided alone or in combination with other active ingredients.

While not being bound by theory, it is believed that the epitope on Ang2 bound by MEDI1/5 maps to the fibronectin domain required for Ang2 binding to the Tie2 receptor and thus, treatment with MEDI1/5 should prevent Ang2-Tie2 interaction. This is further supported by the finding that MEDI1/5 has substantially greater affinity for human Ang2 over human Ang1. Likewise, ex vivo treatment of cancer patient serum with MEDI1/5 has demonstrated suppression of endogenous Ang2 and also endogenous Ang1, albeit higher concentrations of MEDI1/5 were needed to suppress Ang1. In vivo, MEDI1/5 has demonstrated anti-angiogenic and anti-tumor activities in preclinical models. This evidence and the exemplary evidence provided herein supports the methods of treatment disclosed.

B. Methods of Inhibiting Angiogenesis

Another embodiment encompasses a method of inhibiting angiogenesis in a patient comprising
a. providing an anti-Ang2 antibody or functional part thereof,
b. administering an anti-Ang2 antibody or functional part thereof to the patient, wherein the anti-Ang2 antibody or functional part thereof is administered at a dose from about 300 mg to about 1500 mg.

In one embodiment of a method of inhibiting angiogenesis, the patient has cancer.

C. Antibodies for Use in Methods of Treatment

The present methods may use any anti-Ang2 antibody or functional part thereof. In one embodiment, the anti-Ang2 antibody or functional part thereof has the same heavy chain variable region and light chain variable region as MEDI1/5 (SEQ ID NOs: 1 and 2). In another embodiment, the anti-Ang2 antibody or functional part thereof has the same heavy and light chain CDRs as MEDI1/5 (CDRs shown in bold in SEQ ID NOs: 1 and 2). In another mode, the antibody or functional part thereof binds to the same epitope as MEDI1/5. In another aspect, the antibody functional part is a functional part of the antibody MEDI1/5.

In another embodiment, the anti-Ang2 antibody or functional part thereof is disclosed in U.S. Pat. No. 8,507,656, for example col. 11, line 56 through col. 20, which is incorporated by reference in its entirety herein for the description of anti-Ang2 antibodies and functional parts thereof. In one aspect, antibodies or functional parts are capable of binding Ang-2, treating cancer, inhibiting angiogenesis, antagonizing Ang-2 and/or antagonizing Tie-2.

In one embodiment, the antibody or functional part thereof comprises a variable light chain comprising a sequence chosen from 3.19.3 light chain, MEDI1; MEDI2; MEDI3; MEDI4; and MEDI6 as incorporated by reference from U.S. Pat. No. 8,507,656. In one aspect, the antibody or functional part thereof is an IgG1 or an IgG2 isotype antibody or functional part thereof. In another aspect, the antibody or functional part thereof further comprises a variable heavy chain region comprising a sequence chosen from 3.19.3 heavy chain and MEDI5 as incorporated by reference from U.S. Pat. No. 8,507,656.

In one embodiment, the antibody or functional part thereof binds to the same epitope as any one of fully human monoclonal antibodies chosen from 3.19.3, MEDI1/5, MEDI2/5, MEDI3/5, MEDI6/5, and MEDI4/5 as incorporated by reference from U.S. Pat. No. 8,507,656. In another embodiment, the antibody is a fully human monoclonal antibody chosen from: 3.19.3, MEDI1/5, MEDI2/5, MEDI3/5, MEDI6/5, and MEDI4/5 as incorporated by reference from U.S. Pat. No. 8,507,656. In another embodiment, the antibody functional part is a functional part of a fully human monoclonal antibody chosen from: 3.19.3, MEDI1/5, MEDI2/5, MEDI3/5, MEDI6/5, and MEDI4/5 as incorporated by reference from U.S. Pat. No. 8,507,656.

D. Dosing

In either the methods of treating cancer or the methods of inhibiting angiogenesis, varying dosage approaches may be used for the anti-Ang2 antibody or functional part thereof.

In one embodiment, the anti Ang2-antibody or functional part thereof may be administered at a dose from about 200 mg to about 1500 mg, from about 1000 mg to about 1500 mg, from about 750 mg to about 1250 mg, or from about 900 mg to about 1100 mg. In one embodiment, functional part thereof may be administered at a dose of about 200 mg, about 300 mg, about 600 mg, about 750 mg, about 1000 mg, about 1250 mg, or about 1500 mg.

In one embodiment, the anti-Ang2 antibody or functional part thereof is administered an IV infusion over from about 60 to about 90 minutes. In one aspect, IV infusion may be over about 60 minutes and the dosage may be less than about 1000 mg. In another aspect, the IV infusion may be over about 90 minutes and the dosage may be greater than or equal to about 1000 mg.

In one mode, the patient receives one dosage. In another mode, the patient receives multiple doses. In one embodiment, the dosage cycle is one week, two weeks, three weeks, four weeks, five weeks, or six weeks. In one embodiment, the dosage cycle is about every 7 days, about every 14 days, about every 21 or about every 28 days. By a 21 day dosage cycle, for example, we mean receiving the dose on day 1 and then having an additional 20 days of not receiving a dose, followed by receiving the next dose on day 22 and so on.

In one embodiment, a dose of 1500 mg is provided every 21 days. In another embodiment, a dose of 1000 mg is provided every 14 days.

In one embodiment, a dose of from about 300 mg to about 1500 mg is provided every 21 days. In another embodiment, a dose of from about 200 mg to about 1000 mg is provided every 14 days.

In one embodiment, there are at least 2 dosage cycles (i.e., the patient receives two doses). In another embodiment, there are at least 3, 4, 5, 6, 7, 8, 9, 10, 11, 12, or more than 12 dosage cycles. In one embodiment, there are from 12 to 18 dosage cycles. In another embodiment, there are from 12 to 31 dosage cycles.

E. Coadministration with At Least One Additional Therapeutic Agent

The anti-Ang2 antibody or functional part thereof may be administered alone. In another embodiment, the anti-Ang2 antibody or functional part thereof may be coadministered with at least one additional therapeutic agent. In one mode, the anti-Ang2 antibody or functional part is coadministered with two or more additional therapeutic agents. The coadministration may be concurrent administration or sequential administration. The sequential administration may occur on the same day or on different days. If the sequential administration occurs on different days, it may occur on the same dosage cycle or a different dosage cycle.

In one embodiment, at least one additional therapeutic agent is at least one chemotherapeutic agent. In certain aspects, the chemotherapeutic agent may be chosen from at least one of carboplatin, capecitabine, gemcitabine, or paclitaxel. In one aspect, the at least one chemotherapeutic agent is carboplatin and paclitaxel. In one aspect, the at least one chemotherapeutic agent is carboplatin and gemcitabine.

In one embodiment, the dosage cycle for the additional therapeutic agent is three days, one week, two weeks, three weeks, four weeks, five weeks, or six weeks. In one embodiment, the dosage cycle for the additional therapeutic agent is about every 3 days, 7 days, 14 days, 21 days, or about every 28 days.

If the chemotherapeutic agent is paclitaxel, in one embodiment, it may be administered at about 80 mg/m². If the chemotherapeutic agent is paclitaxel, in one embodiment, it may be administered at about 175 mg/m². If the chemotherapeutic agent is gemcitabine, in one embodiment, it may be administered at about 1000 mg/m². If the chemotherapeutic agent is carboplatin, in one embodiment, it may be administered at about AUC 4 or 5.

The AUC-based dosing for carboplatin may be determined using the Follow-Up for Action Letter for Protocols Sponsored by the National Cancer Institute that Use Carboplatin, dated Oct. 14, 2010, which is incorporated by reference in its entirety for carboplatin dosing guidelines. In one embodiment, the Calvert Formula is used, wherein

Total Dose (mg)=(target AUC)×(GFR+25)

and wherein GFR is the glomerular filtration rate. In one embodiment, the GFR is estimated by using the serum creatinine level. In one aspect, the maximum carboplatin dose does not exceed the target AUC (mg min/mL)×150 mL/min. For example, in one embodiment, the maximum carboplatin dose may be about 750 mg for an AUC of 5 and about 600 mg for an AUC of 4. Alternatively, in another embodiment, such as a patient with low muscle mass, GFR may be measured directly or a minimum creatinine level of 0.6 mg/dL may be used.

In another aspect, at least one additional therapeutic agent is an antibody or functional part thereof. For example, the antibody or functional part thereof may be chosen from an anti-VEGF antibody or functional part thereof. In one mode, the antibody or functional part thereof may be chosen from bevacizumab. In one mode, the bevacizumab is administered at about 10 mg/kg or about 15 mg/kg. In one mode, the bevacizumab may be administered at from about 10 mg/kg to about 15 mg/kg. In one aspect, the bevacizumab may be administered every two weeks or every three weeks.

In another aspect, another agent may be chosen that inhibits VEGF or the VEGF pathway. For example, at least one additional therapeutic agent may be cediranib, an inhibitor of vascular endothelial growth factor receptor.

F. Candidates for Treatment

In one method, the patient has cancer. In one embodiment, the patient has ovarian cancer. In another embodiment, the patient has glioblastoma multiforme.

In one method, the cancer is breast cancer, colon cancer, lung cancer, renal cancer, prostate cancer, ovarian cancer, cervical cancer, liver cancer, gastric cancer, bladder cancer, skin cancer, leukemia, or brain cancer. In another embodiment, the skin cancer is melanoma, the brain cancer is glioma, the brain cancer is glioblastoma multiforme, or the lung cancer is non-small cell lung cancer.

In another method, the cancer is biliary (cholangiocarcinoma), bladder, blood, bone, brain, breast, central nervous system cancer, chest, colon, colorectal, endometrial cancer, epidermoid carcinoma, esophageal, eye, gastroesophageal, glioblastoma, glioma, head and neck, kidney, laryngeal, leukemia, liver (such as hepatocellular carcinoma), lung, lymph nodes, lymphoma, melanoma, mesothelioma, mouth, myeloma, non-small cell lung carcinoma, ovary, pancreas, pediatric malignancies, prostate, rectum, salivary gland, sarcoma, small bowel adenocarcinoma, small cell lung carcinoma, stomach, testes, throat, thyroid, and/or uterus.

Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple mycloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as but not limited to, glioma, astrocytoma, brain stem glioma, ependymoma, oligodendroglioma, non-glial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or ureter); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesotheliorna, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

The Karnofsky performance status index allows patients to be classified as to their functional impairment. The lower the Karnofsky score, the worse the survival for most serious illnesses.

TABLE 2
Karnofsky Performance Status

| | Karnofsky | |
|---|---|---|
| Category | Status | Description |
| Able to carry on normal | 100 | Normal no complaints; no |
| activity and to work; no | | evidence of disease. |
| special care needed | 90 | Able to carry on normal |
| | | activity; minor signs or |
| | | symptoms of disease. |
| | 80 | Normal activity with effort; |
| | | some signs or symptoms of |
| | | disease. |
| Unable to work; able to | 70 | Cares for self; unable to carry |
| live at home and care for | | on normal activity or to do |
| most personal needs; | | active work. |
| varying amount of | 60 | Requires occasional |
| assistance needed. | | assistance, but is able to care |
| | | for most of his personal needs. |
| | 50 | Requires considerable |
| | | assistance and frequent |
| | | medical care. |
| Unable to care for self; | 40 | Disabled; requires special care |
| requires equivalent of | | and assistance. |
| institutional or hospital | 30 | Severely disabled; hospital |
| care; disease may be | | admission is indicated |
| progressing rapidly. | | although death not imminent. |
| | 20 | Very sick; hospital admission |
| | | necessary; active supportive |
| | | treatment necessary. |
| | 10 | Moribund; fatal processes |
| | | progressing rapidly. |
| | 0 | Dead |

In one embodiment, the patient has a Karnofsky performance status greater than or equal to about 60. In another embodiment, the patient has a Karnofsky performance status greater than or equal to about 70.

### II. Nucleic Acids Encoding Antibodies and Functional Parts Thereof

A. Methods of Use of Nucleic Acids Encoding Antibodies or Functional Parts

In yet another embodiment a nucleic acid encoding an antibody or functional part may be administered. Upon administration of such nucleic acid, antibodies or functional parts are produced by the host's machinery. In one aspect, produced antibodies or functional parts are capable of binding Ang-2, treating cancer, inhibiting angiogenesis, antagonizing Ang-2 and/or antagonizing Tie-2.

A nucleic acid encoding a functional part of an antibody refers a nucleic acid at least 30 base pairs long, at least 50 base pairs long, or at least 100 base pairs long, comprising at least one expression characteristic (in kind not necessarily in amount) as a nucleic acid encoding an antibody. In one embodiment, a nucleic acid encoding a functional part of an antibody at least encodes an amino acid sequence comprising two or optionally three CDRs of the antibodies described herein.

### III. Methods of Making Antibodies and Functional Parts

An isolated antibody producing cell capable of producing an antibody or functional part is also provided. Certain methods of producing an antibody or functional part thereof are provided in U.S. Pat. No. 8,507,656, for example col. 21, line 4 through col. 25, line 27, which is incorporated by reference in its entirety herein for the description of methods of making antibodies and functional parts thereof.

The antibodies or functional parts described herein may be manufactured from a hybridoma that secretes the antibody or functional part thereof or from a recombinantly produced cell that has been transformed or transfected with a gene or genes encoding the antibody or functional part.

One embodiment includes a method of producing the antibody or functional part by culturing host cells under conditions wherein a nucleic acid is expressed to produce the antibody or functional part thereof, followed by recovering the antibody or functional part thereof. A variety of cell lines may be used for expressing the antibody or functional part, including, but not limited to, mammalian cell lines. In one embodiment, the cell lines may be human. In another embodiment, bacterial or insect cell lines may be used. In one embodiment, the cell lines include Chinese hamster ovary (CHO) cells, variants of CHO cells (for example DG44), 293 cells and NSO cells. In another embodiment, cell lines include VERY, BHK, Hela, COS, MDCK, 293F, 293T, 3T3, W138, BT483, Hs578T, HTB2, BT20 and T47D, CRL7O3O and HsS78Bst cells.

Recombinant expression utilizes construction of an expression vector containing a polynucleotide that encodes the antibody or functional part. Once a polynucleotide has been obtained, a vector for the production of the antibody or functional part thereof may be produced by recombinant DNA technology well known in the art. Expression vectors may include appropriate transcriptional and translational control signals. This may be accomplished using in vitro recombinant DNA techniques, synthetic techniques, and in vivo genetic recombination. In one embodiment, a replicable vector comprises a nucleic acid sequence encoding an antibody or functional part operably linked to a heterologous promoter.

A variety of host-expression vector systems may be utilized to express antibodies or functional parts as described in U.S. Pat. No. 5,807,715. For example, mammalian cells such as Chinese hamster ovary cells (CHO), in conjunction with a vector such as the major intermediate early gene promoter element from human cytomegalovirus, are an effective expression system for antibodies (Foecking et al., Gene, 45:101 (1986); and Cockett et al., Bio/Technology, 8:2 (1990)). In addition, a host cell strain may be chosen which modulates the expression of inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the protein of the invention. To this end, eukaryotic host cells which possess the cellular machinery for proper processing of the primary transcript, glycosylation, and phosphorylation of the gene product may be used.

In bacterial systems, a number of expression vectors may be selected depending upon the use intended for the antibody or functional part being expressed. For example, when a large quantity of such an antibody or functional part is to be produced, for the generation of pharmaceutical compositions comprising an antibody or functional part, vectors which direct the expression of high levels of fusion protein products that are readily purified may be desirable. Such vectors include, but are not limited to, the E. coli expression vector pUR278 (Ruther et al., EMBO, 12:1791 (1983)), in which the coding sequence may be ligated individually into the vector in frame with the lac Z coding region so that a fusion protein is produced; pIN vectors (Inouye & Inouye, 1985, Nucleic Acids Res. 13:3101-3109 (1985); Van Heeke & Schuster, 1989, J. Biol. Chem., 24:5503-5509 (1989)); and the like. pGEX vectors may also be used to express foreign polypeptides as fusion proteins with glutathione-S-transferase (GST). In general, such fusion proteins are soluble and can easily be purified from lysed cells by adsorption and binding to glutathione-agarose affinity matrix followed by elution in the presence of free glutathione. The pGEX vectors are designed to introduce a thrombin and/or factor Xa protease cleavage sites into the expressed polypeptide so that the cloned target gene product can be released from the GST moiety.

In an insect system, Autographa californica nuclear polyhedrosis virus (AcNPV) is used as a vector to express foreign genes. The virus grows in Spodoptera frugiperda cells. The protein coding sequence may be cloned individually into non-essential regions (for example, the polyhedrin gene) of the virus and placed under control of an AcNPV promoter (for example, the polyhedrin promoter).

In mammalian host cells, a number of virus based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion into a non-essential region of the viral genome (e.g., region E1 or E3) will result in a recombinant virus that is viable and capable of expressing the antibody or functional part in infected hosts (e.g., see, Logan & Shenk, Proc. Natl. Acad. Sci. USA, 81:355-359 (1984)). Specific initiation signals may also be required for efficient translation of inserted antibody or functional part coding sequences. These signals include the ATG initiation codon and adjacent sequences. Furthermore, the initiation codon should generally be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol., 153:51-544(1987)).

Stable expression can be used for long-term, high-yield production of recombinant proteins. For example, cell lines which stably express the protein molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Plasmids that encode an antibody or functional part can be used to introduce the gene/cDNA into any cell line suitable for production in culture.

A number of selection systems may be used, including, but not limited to, the herpes simplex virus thymidine kinase (Wigler et al., Cell, 11:223 (1977)), hypoxanthineguanine phosphoribosyltransferase (Szybalska & Szybalski, Proc. Natl. Acad. Sci. USA, 48:202 (1992)), and adenine phosphoribosyltransferase (Lowy et al., Cell, 22:8-17 (1980)) genes can be employed in tk-, hgprt- or aprT-cells, respectively. Also, antimetabolite resistance can be used as the basis of selection for the following genes: dhfr, which confers resistance to methotrexate (Wigler et al., Natl. Acad. Sci. USA, 77:357 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol., 150:1.

Once an antibody or functional part has been produced by recombinant expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the proteins of the present invention or fragments thereof may be fused to heterologous polypeptide sequences described herein or otherwise known in the art to facilitate purification.

Reference will now be made in detail to the present exemplary embodiments, examples of which are illustrated in the accompanying drawings. Wherever possible, the same reference numbers will be used throughout the drawings to refer to the same or like parts. Other embodiments will be apparent to those skilled in the art from consideration of the specification and practice disclosed herein. The embodiments are further explained in the following examples. These examples do not limit the scope of the claims, but merely serve to clarify certain embodiments. It is intended that the specification and examples be considered as exemplary only, with a true scope and spirit being indicated by the following claims.

### EXAMPLES

### Example 1

### Safety, Tolerability, Antitumor Activity, and Pharmacology Study

This is a first time in human, Phase 1/1b, multicenter, open-label, single-arm, dose-escalation and dose-expansion study of MEDI1/5 to evaluate the safety, tolerability, antitumor activity, and pharmacology of MEDI1/5 as a single agent or in combination therapy in adult subjects with advanced solid tumors refractory to standard therapy or for which no standard therapy exists.

Approximately 5-15 investigational sites in the United States will participate in the dose-escalation and dose-expansion arms of the study. Based on each site's Institutional Review Board (IRB) regulatory approval, sites may enroll subjects into the Phase 1 or Phase 1b dose-escalation and dose-expansion arms, or both phases of the study.

The Phase 1 study is a 3+3 dose escalation (monotherapy [mTx] and combination) in adults with advanced solid tumors with mTx expansion into platinum-resistant ovarian cancer (prOC) and glioblastoma multiforme (GBM) (NCT01248949). Patients with Karnofsky performance status ≧70 (or ≧60 for patients with glioblastoma), and adequate organ function were treated in 21 or 28 day cycles with MEDI1/5 (M) alone or in combination with carboplatin/paclitaxel (CT), paclitaxel (T), or bevacizumab (B). Objectives included safety, pharmacokinetics, pharmacodynamics, and antitumor activity.

In the Phase 1 dose-escalation arms, subjects were treated with 1 of 7 doses of MEDI1/5 (5, 10, 20, 100, 300, 1000, or 1500 mg) every 14 or 21 days, depending on the cycle. At the discretion of the sponsor, an intermediate dose could be chosen for dose escalation. MEDI1/5 was administered on Day 1 of each cycle of treatment as a 60-minute IV infusion for doses less than 1000 mg, or 90-minute infusion for doses 1000 mg and greater (to reduce the potential for infusion reactions at higher doses), until unacceptable toxicity, documentation of disease progression, or other reasons for subject discontinuation. Intra-subject dose-escalation was not allowed, but dose modification for toxicities was allowed.

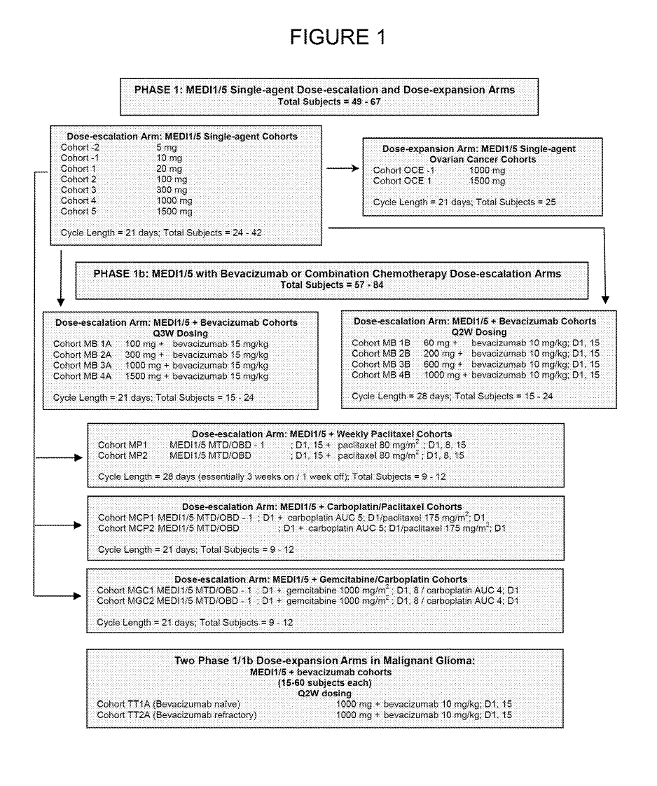

The overall study design is outlined in FIG. 1.

### Example 2

### Results from Ongoing Study

As of 25 Apr. 2014, 104 patients (median age 61.5; 57.7% female) were enrolled: 41 in M mTx (25 in dose-escalation and 16 in prOC dose-expansion), and 63 in combination arms. Table 3 provides patient demographics and baseline characteristics, including median age, gender, KPS, site of primary disease, and stage at diagnosis.

TABLE 3
Patient Demographics and Baseline Characteristics

| | MEDI1/5 Monotherapy, n (%) | | | MEDI1/5 Combinations, n (%) | |
|---|---|---|---|---|---|
| | MEDI1/5 | Ovarian Cancer | MEDI1/5 | MEDI1/5 + | MEDI1/5 + |
| | Monotherapy | Monotherapy | Monotherapy | Bevacizumab | Chemotherapy |
| | Escalation Arm | Expansion Arm | All Arm | Arm | Arm* |
| Variable | (n = 25) | (n = 16) | (n = 41) | (n = 43) | (n = 20) |
| Median age, y | 63 (21-81) | 60.5 (27-79) | 63 (21-81) | 59 (25-75) | 60.5 (31-79) |
| (range) | | | | | |
| Female, n (%) | 10 (40) | 16 (100) | 26 (63) | 21 (49) | 13 (65) |

KPS, n (%)

| | | | | | |
|---|---|---|---|---|---|
| 70-80 | 9 (36) | 3 (23) | 12 (29) | 22 (51) | 12 (60) |
| 90-100 | 16 (64) | 10 (77) | 26 (68) | 21 (49) | 8 (40) |

Site of primary disease, n (%)

| | | | | | |
|---|---|---|---|---|---|
| Ovarian | 3 (12) | 16 (100) | 19 (46) | 10 (23) | 5 (25) |
| Non-small cell | 11 (44) | 0 | 11 (27) | 1 (2) | 3 (15) |
| lung | | | | | |
| Other | 7 (28) | 0 | 7 (17) | 10 (23) | 5 (25) |
| Colon/colorectal | 3 (12) | 0 | 3 (7) | 6 (14) | 0 |
| Renal cell | 1 (4) | 0 | 1 (2) | 3 (7) | 0 |
| carcinoma | | | | | |
| Esophageal/ | 0 | 0 | 0 | 3 (7) | 1 (5) |
| gastroesophageal | | | | | |
| Endometrial | 0 | 0 | 0 | 2 (5) | 0 |
| Hepatocelluar | 0 | 0 | 0 | 2 (5) | 0 |
| Glioblastoma | 0 | 0 | 0 | 2 (5) | 0 |
| Prostate | 0 | 0 | 0 | 1 (2) | 0 |
| Laryngeal | 0 | 0 | 0 | 1 (2) | 0 |
| Pancreatic | 0 | 0 | 0 | 1 (2) | 0 |
| Cervical | 0 | 0 | 0 | 1 (2) | 0 |
| Thyroid | 0 | 0 | 0 | 0 | 2 (10) |
| Melanoma | 0 | 0 | 0 | 0 | 1 (5) |
| Testicular | 0 | 0 | 0 | 0 | 1 (5) |

Stage at diagnosis, n (%)

| | | | | | |
|---|---|---|---|---|---|
| I | 0 | 0 | 0 | 1 (2) | 2 (10) |
| II | 0 | 0 | 0 | 1 (2) | 0 |
| III | 4 (16) | 12 (75) | 16 (39) | 7 (16) | 7 (35) |
| IV | 21 (84%) | 3 (19%) | 24 (59%) | 31 (72%) | 11 (55%) |

The maximum tolerated dose was not defined in either mTx or combination arms.

In the mTx arms, 27 of 41 patients (66%) of patients had treatment-related adverse events (trAEs). All 27 patients (66%) experienced at least one grade ≦2 event and 4 patients (10%) experienced at least one grade ≧3 event. Non-hematologic grade ≧3 trAEs in the mTx arms included increased weight (4.9%), peripheral edema (3.1%), lymphedema (2.4%), pleural effusion (2.4%), hypertension (2.4%), and posterior reversible encephalopathy syndrome (2.4%). The grade 3 peripheral edema and lymphedema that occurred in 2 patients in the prOC expansion arm persisted despite discontinuation of M.

In the combination arms, 49 of 63 patients (78%) had treatment-related adverse events (trAEs). This includes 45 patients (71%) with at least one grade ≦2 event and 14 patients (22%) with at least one grade ≧3 event. Non-hematologic grade ≧3 trAEs in the combination arms included one patient (1.6%) of each of the following: nausea, acute pancreatitis, vomiting, fatigue, peripheral edema, infusion related reaction, decreased ejection fraction, increased troponins, decreased appetite, dehydration, peripheral neuropathy, nephrotic syndrome, female genital tract fistula, and scrotal edema. Additionally, two patients (3.2%) experienced proteinuria and four patients (6.3%) experienced hypertension.

The trAE events (heme and non-heme) are summarized by total number of patients in the table below:

TABLE 4
Treatment-Related Adverse Events (trAE)

| | MEDI1/5 | MEDI1/5 Combination | | MEDI1/5 |
|---|---|---|---|---|
| | Single Agent | +Bev | +Chemo | Total |
| trAE | (N = 41) | (N = 43) | (N = 20) | (N = 104) |
| Grade ≦2 (%) | 27 (65.9%) | 28 (65.1%) | 17 (85.0%) | 72 (69.2%) |
| Grade ≧3 (%) | 4 (9.8%) | 9 (20.9%) | 5 (25.0%) | 18 (17.3%) |
| Total (%) | 27 (65.9%) | 32 (74.4%) | 17 (85.0%) | 76 (73.1%) |

The non-hematologic grade ≧3 events are summarized as follows for mTx and combination treatment by total number of events in the table below:

TABLE 5
Non-hematologic Grade ≧3 events

| | | MEDI1/5 | MEDI1/5 Combination | | MEDI1/5 |
|---|---|---|---|---|---|
| | | Single Agent | +Bev | +Chemo | Total |
| trAE | Grade | (N = 41) | (N = 43) | (N = 20) | (N = 104) |
| Appetite decreased | 3 | 0 (0%) | 0 (0%) | 1 (5.0%) | 1 (1.0%) |
| Dehydration | 3 | 0 (0%) | 0 (0%) | 1 (5.0%) | 1 (1.0%) |
| Ejection fraction decreased | 3 | 0 (0%) | 0 (0%) | 1 (5.0%) | 1 (1.0%) |
| Fatigue | 4 | 0 (0%) | 0 (0%) | 1 (5.0%) | 1 (1.0%) |
| Female genital fistula | 3 | 0 (0%) | 1 (2.3%) | 0 (0%) | 1 (1.0%) |
| Hypertension | 3 | 1 (2.4%) | 4 (9.3%) | 0 (0%) | 5 (4.8%) |
| Infusion related reaction | 3 | 0 (0%) | 0 (0%) | 1 (5.0%) | 1 (1.0%) |
| Leukopenia | ≧3 | 0 (0%) | 0 (0%) | 1 (5.0%) | 1 (1.0%) |
| Lymphedema | 3 | 1 (2.4%) | 0 (0%) | 0 (0%) | 1 (1.0%) |
| Nausea | 3 | 0 (0%) | 0 (0%) | 1 (5.0%) | 1 (1.0%) |
| Nephrotic syndrome | 3 | 0 (0%) | 0 (0%) | 1 (5.0%) | 1 (1.0%) |
| Neutropenia | c | 1 (2%) | 0 (0%) | 3 (15%) | 4 (3.8%) |
| Pancreatitis acute | 3 | 0 (0%) | 1 (2.3%) | 0 (0%) | 1 (1.0%) |
| Peripheral edema | 3 | 1 (2.4%) | 1 (2.3%) | 0 (0%) | 2 (1.9%) |
| Peripheral neuropathy | 3 | 0 (0%) | 1 (2.3%) | 0 (0%) | 1 (1.0%) |
| Pleural effusion | 3 | 1 (2.4%) | 0 (0%) | 0 (0%) | 1 (1.0%) |
| Posterior reversible encephalopathy | 3 | 1 (2.4%) | 0 (0%) | 0 (0%) | 1 (1.0%) |
| Proteinuria | 3 | 0 (0%) | 2 (4.7%) | 0 (0%) | 2 (1.9%) |
| Scrotal edema | 3 | 0 (0%) | 1 (2.3%) | 0 (0%) | 1 (1.0%) |
| Troponins increased | 3 | 0 (0%) | 0 (0%) | 1 (5.0%) | 1 (1.0%) |
| Vomiting | 3 | 0 (0%) | 0 (0%) | 1 (5.0%) | 1 (1.0%) |
| Weight increased | 3 | 2 (4.9%) | 0 (0%) | 0 (0%) | 2 (1.9%) |
| White blood cell count decreased | ≧3 | 1 (2.4%) | 0 (0%) | 0 (0%) | 1 (1.0%) |
| Total | — | 9 (22.0%) | 11 (25.6%) | 13 (65%) | 33 (31.7%) |

The rate of treatment-related grade 3 or 4 adverse events was 22% in the monotherapy group and 38% overall in the combination groups.

Six patients (14.6%) in the mTx arms, 23 patients (36.5%) in the combination arms and 29 patients (27.9%) overall discontinued treatment due to trAEs. Some of the adverse events leading to discontinuation included neutropenia, peripheral edema, increased weight, acute pancreatitis, generalized edema, pyrexia, proteinurea, female genital tract fistula, infusion-related reaction, decreased ejection fraction, decreased troponin, nephrotic syndrome, and joint swelling.

Treatment-related grade 3 or 4 adverse events with MEDI1/5 were also compared to historical bevacizumab data in Table 6.

TABLE 6
Comparison of Treatment-Related Grade 3 or 4 Adverse Events
with MEDI1/5 Versus Historical Bevacizumab Data

| | MEDI1/5 | MEDI1/5 + | MEDI1/5 + | Bevaciz- |
|---|---|---|---|---|
| Adverse | Monotherapy | Bevaciz- | Chemother- | umab† |
| Event, % | All Arms | umab Arm | apy Arm* | (historical |
| of patients | (n = 41) | (N = 43) | (n = 20) | rates) |
| Hypertension | 2.4% | 9.3% | 0 | 5%-18% |
| Proteinuria | 0 | 4.7% | 0 | ≦8% |
| Female genital | 0 | 2.3% | 0 | Non-GI |
| tract fistula (no | | | | fistula <1% |
| other fistulas) | | | | |
| Peripheral | 2.4% | 2.3% | 0 | N/A |
| edema | | | | |
| Increased | 4.9% | 0 | 0 | N/A |
| weight | | | | |
| Lymphedema | 2.4% | 0 | 0 | N/A |
| Pleural | 2.4% | 0 | 0 | N/A |
| effusion | | | | |

*Includes MEDI1/5 in combination with paclitaxel and carboplatin/paclitaxel
†Per Avastin ™ prescribing information

Because monotherapy with MEDI1/5 at a dose of 1500 mg Q3W resulted in grade 3 edema-related toxicities in the ovarian cancer subsets, a dose of 1000 mg Q3W was selected for subsequent ovarian cancer patients. Edema-related events of this nature were not observed in patients with other tumor types. The bevacizumab combination had an acceptable safety profile.

Patient exposure data for mTx and combination arms are shown in the table below:

TABLE 7
Patient Exposure Data

| | | |
|---|---|---|
| MEDI1/5 | MEDI1/5 | MEDI1/5 |
| Single Agent | Combination | Total |
| (N = 41) | (N = 63) | (N = 104) |

Total Number of Cycles

| | | | |
|---|---|---|---|
| Mean | 3.4 | 4.6 | 4.1 |
| Median | 2.0 | 2.0 | 2.0 |
| Min-Max | 1-18 | 1-35 | 1-31 |

Total Number of Cycles

| | | | |
|---|---|---|---|
| 1 | 11 | 13 | 24 |
| 2 | 16 | 26 | 42 |
| 3 | 1 | 2 | 3 |
| 4-6 | 9 | 14 | 23 |
| 7-9 | 1 | 1 | 2 |
| 10-12 | 1 | 0 | 1 |
| >12 | 2 | 7 | 9 |

Dose Intensity (%)

| | | | |
|---|---|---|---|
| Mean | 96.26 | 95.21 | 95.62 |
| Median | 100.0 | 100.0 | 100.0 |
| Min-Max | 66.7-100.0 | 45.9-100.0 | 45.9-100.0 |

DOSE INTENSITY (%) = (TOTAL ACTUAL DOSE RECEIVED/TOTAL DOSE INTENDED) × 100.

Exposure of M approached a linear range beyond 100 mg Q3W or 60 mg Q2W.

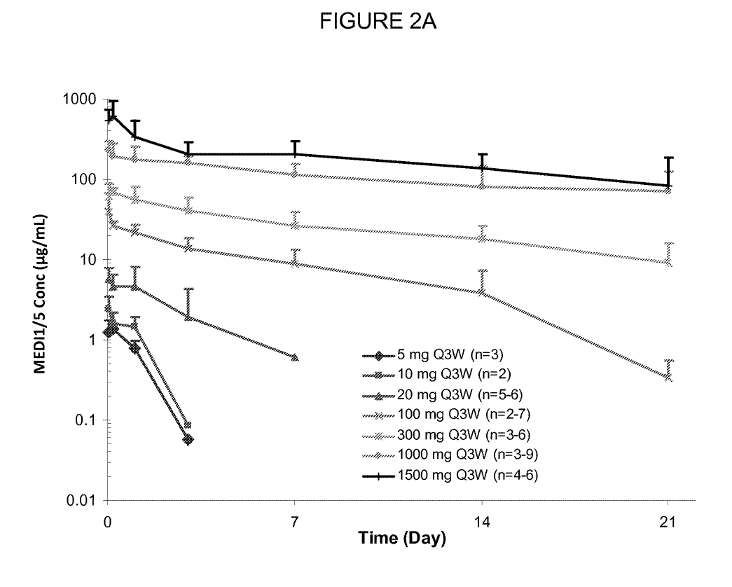

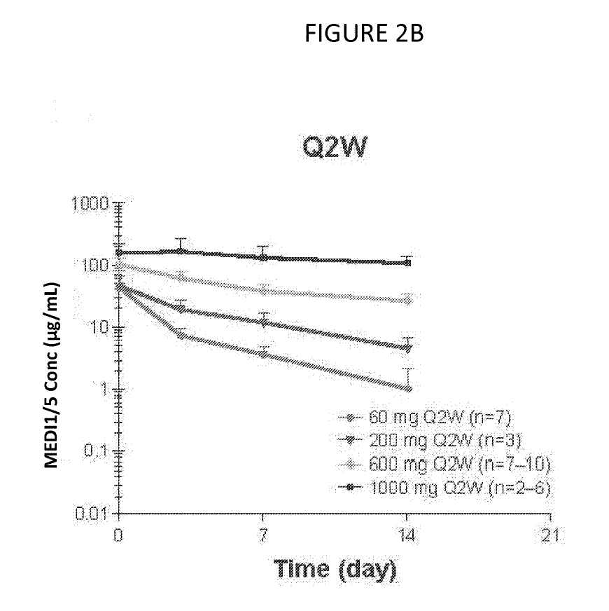

Interim pharmacokinetic analysis was performed based on PK data collected as of 2 Jan. 2013. A validated immunoassay was used to quantify serum concentrations of MEDI1/5 that does not bind to its target. The serum MEDI1/5 PK was determined for single-agent therapy in Phase 1 as well as combination therapy in Phase 1b. The MEDI1/5 PK was generally comparable across single-agent or combination therapy for the same dose regimen (data not shown). Therefore, the mean serum MEDI1/5 concentration-time profiles are summarized from all subjects pooled by dose regimen. The mean serum MEDI1/5 concentration-time profiles during the first dose (Day 1 to Day 22 of Q3W regimen, Day 1 to Day 15 of Q2W regimen) after IV administrations of MEDI1/5 at 5, 10, 20, 100, 300, 1000, and 1500 mg are illustrated in FIGS. 2A-B. Mean serum concentrations increased with an increase of MEDI1/5 dose levels.

The serum concentration data from the first dosing were analyzed using non-compartmental analysis. The exposure of MEDI1/5 based on Cₘₐₓ and AUC after the first dose demonstrated a more than dose-proportional increase. Dose-dependent apparent clearance and terminal half-life were also observed. MEDI1/5 PK approached a linear range approximately beyond 100 mg Q3W or 60 mg Q2W. The estimated mean PK parameters are presented in the table below.

TABLE 8
Summary of MEDI1/5 Pharmacokinetic Parameters

| Dosing | Dose | Cₘₐₓ | AUC_{(0-τ)} | CL | t_{1/2} |
|---|---|---|---|---|---|
| Interval | (mg) | (μg/mL) | (day · μg/mL) | (L/day) | (day) |
| Q3W | 5 | 1.39 ± 0.25 | 2.17 | 2.25 | 0.625 |
| | | (n = 3) | (n = 1) | (n = 1) | (n = 1) |
| | 10 | 2.44 ± 0.96 | 1.73 | 3.52 | 0.703 |
| | | (n = 3) | ( )n = 1) | (n = 1) | (n = 1) |
| | 20 | 6.63 ± 3.48 | 13.7 ± 15.4 | 2.32 ± 1.49 | 1.10 ± 0.33 |
| | | (n = 6) | (n = 5) | (n = 5) | (n = 5) |
| | 100 | 39.7 ± 26.4 | 156 ± 68.9 | 0.622 ± 0.374 | 5.22 ± 3.04 |
| | | (n = 7) | (n = 6) | (n = 6) | (n = 6) |
| | 300 | 68.9 ± 16.9 | 517 ± 176 | 0.551 ± 0.259 | 8.14 ± 2.76 |
| | | (n = 6) | (n = 5) | (n = 5) | (n = 5) |
| | 1000 | 223 ± 61.8 | 1813 ± 506 | 0.423 ± 0.193 | 12.0 ± 5.47 |
| | | (n = 10) | (n = 8) | (n = 8) | (n = 8) |
| | 1500 | 578 ± 294 | 3574 ± 2088 | 0.424 ± 0.251 | 12.8 ± 10.3 |
| | | (n = 7) | (n = 4) | (n = 4) | (n = 4) |
| Q2W | 60 | 44.5 ± 39.3 | 118 ± 68.7 | 0.704 ± 0.487 | 3.93 ± 2.77 |
| | | (n = 7) | (n = 7) | (n = 7) | (n = 7) |
| | 200 | 47.5 ± 23.3 | 227 ± 66.3 | 0.815 ± 0.239 | 5.10 ± 0.45 |
| | | (n = 3) | (n = 3) | (n = 3) | (n = 3) |
| | 600 | 107 ± 29.5 | 627 ± 225 | 0.740 ± 0.269 | 7.06 ± 1.27 |
| | | (n = 11) | (n = 8) | (n = 8) | (n = 8) |
| | 1000 | 167 ± 60.4 | N/A | N/A | N/A |
| | | (n = 2) | | | |

Cₘₐₓ = maximum serum concentration; AUC_{(0-τ)} = area under the curve from time zero to dosing interval; CL = apparent systemic clearance; t_{1/2} = terminal half-life; N/A = not applicable.
Note:
Table reflects data collected as of 2 Jan. 2013. Parameters are presented as mean ± standard deviation (sample size)

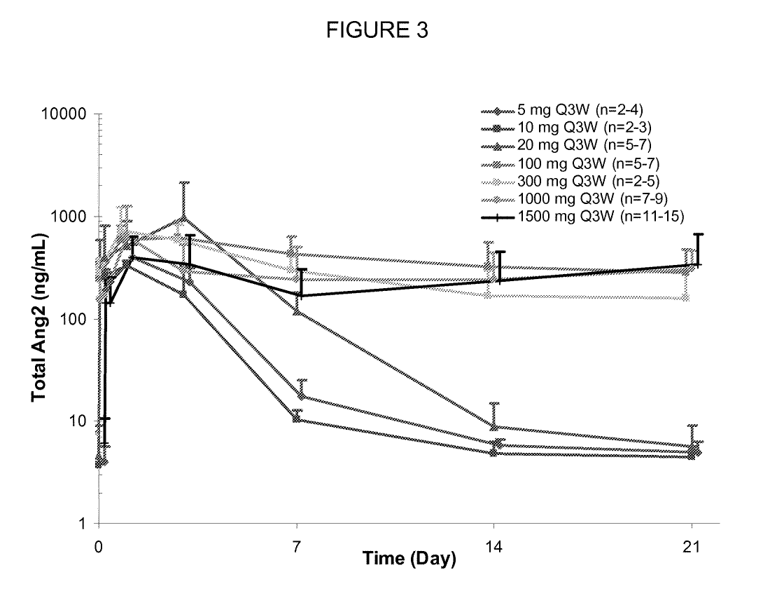

Plasma concentrations of total Ang2, free and drug bound, were quantified as a pharmacodynamics biomarker using a qualified immunoassay. Dose dependent increase in total Ang2 concentrations were observed following MEDI1/5 administration (FIG. 3). Total Ang2 concentration increased rapidly and reached saturating level at all doses tested. The duration of Ang2 accumulation was dose dependent. At doses of 100 mg Q3W or 60 mg Q2W (data not shown) and above, the high concentrations of total Ang2 were maintained throughout the dosing interval. Total Ang2 accumulation reached steady state after the third dose. The Ang2 profiles are consistent with MEDI1/5 PK profiles.

For the purpose of this study, an unconfirmed partial response (PRu) is defined as a partial response on one assessment which was not confirmed on the subsequent assessment after four weeks. The best overall responses achieved in each treatment group are shown in Table 9.

While Table 9 provides data on patients enrolled in the study with a variety of types of cancers, additional disease-specific information is as follows. In platinum-resistant ovarian cancer patients (escalation and expansion arms), the overall response rate for monotherapy was 12% (n=34). One ovarian cancer patient had stable diseases lasting ≧52 weeks (MEDI1/5 and bevacizumab arm) and one ovarian cancer patient had partial response lasting ≧52 weeks (MEDI1/5 and paclitaxel arm). Responses were also observed in patients with lung cancer, cervical cancer, and renal cell carcinoma (one response in each) in the combination therapy arms.

TABLE 9
Best Overall Response

| MEDI1/5 Monotherapy | | | | MEDI1/5 Combination Therapy | |
|---|---|---|---|---|---|
| | MEDI1/5 | Ovarian Cancer | MEDI1/5 | MEDI1/5 + | MEDI1/5 + |
| | Monotherapy | Monotherapy | Monotherapy | Bevacizumab | Chemotherapy |
| Response | Escalation Arm | Expansion Arm | All Arms | Arm | Arm* |
| (RECIST v1.1) | (n = 25) | (n = 16) | (n = 41) | (n = 43) | (n = 20) |
| No. patients | 24 | 9 | 33 | 35 | 16 |
| with baseline | | | | | |
| mid ≧1 on- | | | | | |
| treatment scan | | | | | |
| Complete | 0 | 0 | 0 | 1 (2) | 0 |
| response, n (%) | | | | | |
| Partial response/ | 0 | 1 (6) | 1 (2) | 4 (9) | 3 (15) |
| unconfirmed | | | | | |
| partial response, | | | | | |
| n (%) | | | | | |
| Stable | 13 (52) | 3 (19) | 16 (39) | 18 (42) | 6 (30) |
| disease ≧12 | | | | | |
| weeks, † n | | | | | |
| (%) | | | | | |
| Stable | 1 (4) | 0 | 1 (2) | 2 (5) | 1 (5) |
| disease ≧52 | | | | | |
| weeks, n (%) | | | | | |
| Progressive | 11 (44) | 5 (31) | 16 (39) | 12 (28) | 7 (35) |
| disease, n (%) | | | | | |

*Includes MEDI1/5 combination with paclitaxes and carboplatin/paclitaxel
† Ovarian cancer patient had stable disease lasting ≧52 weeks in MEDI1/5 and bevacizumab arm, and one ovarian cancer patient had partial response lasting ≧52 weeks in the MEDI1/5 and paclitaxel arm.

In the mTx arms (n=41), 1 partial response in a prOC patient was observed and 7 patients had stable disease for >12 weeks.

Objective responses in the combination arms (n=63) included six confirmed partial responses (PR). These responses were seen in the following cohorts: platinum-refractory ovarian cancer (1 PR each in M/B, M/T, and M/CT), renal cell cancer (1 PR in M/B), cervical cancer (1 PR in M/B), lung cancer (1 PR in M/T). Additionally, there was 1 unconfirmed partial responses (PRu) and one confirmed complete response (CR) in GBM (n=3), with one unconfirmed partial response and the confirmed complete response both in MEDI1/5 and bevacizumab combination and one unconfirmed partial response in the MEDI1/5 and chemotherapy arm.

Further information on certain objective responses is provided in Table 10.

TABLE 10
Objective Responses

| | | MEDI1/5 | Additional |
|---|---|---|---|
| | | Dose and | Therapeutic Agent |
| Response | Cancer Type | Cycle | Dosage and Cycle |
| Partial | platinum- | 1000 mg Q3wk | None |
| Response | refractory ovarian | | |
| | cancer | | |
| Partial | platinum- | 1500 mg Q3wk | Bevacizumab 15 |
| response | refractory ovarian | | mg/kg Q3wk |
| | cancer | | |
| Partial | platinum- | 1000 mg Q2wk | Paclitaxel 80 |
| response | refractory ovarian | | mg/m² D 1, 8, 15 |
| | cancer | | Q4wk |
| Partial | renal cell cancer | 200 mg Q2wk | Bevacizumab 10 |
| response | | | mg/kg Q2wk |
| Partial | cervical cancer | 600 mg Q2wk | Bevacizumab 10 |
| response | | | mg/kg Q2wk |
| Partial | lung cancer | 600 mg Q2wk | Paclitaxel 80 |
| response | | | mg/m² D 1, 8, 15 |
| | | | Q4wk |
| Unconfirmed | platinum- | 1000 mg Q3wk | carboplatin |
| partial response | refractory ovarian | | AUC 5 Q3wk |
| | cancer | | paclitaxel 175 |
| | | | mg/m² Q3wk |
| Unconfirmed | malignant glioma | 1000 mg Q2wk | Bevacizumab 10 |
| partial response | | | mg/kg Q2wk |
| Complete | malignant glioma | 1000 mg Q2wk | Bevacizumab 10 |
| Response | | | mg/kg Q2wk |
| Stable disease | platinum- | 5 mg Q3wk | None |
| for 12 weeks | refractory ovarian | | |
| | cancer | | |
| Stable disease | platinum- | 5 mg Q3wk | None |
| for 12 weeks | refractory ovarian | | |
| | cancer | | |
| Stable disease | platinum- | 20 mg Q3wk | None |
| for 12 weeks | refractory ovarian | | |
| | cancer | | |
| Stable disease | platinum- | 20 mg Q3wk | None |
| for 12 weeks | refractory ovarian | | |
| | cancer | | |
| Stable disease | platinum- | 20 mg Q3wk | None |
| for 12 weeks | refractory ovarian | | |
| | cancer | | |
| Stable disease | platinum- | 100 mg Q3wk | None |
| for 12 weeks | refractory ovarian | | |
| | cancer | | |
| Stable disease | platinum- | 300 mg Q3wk | None |
| for 12 weeks | refractory ovarian | | |
| | cancer | | |

### Example 3

### Brain Scan Results

Brain scan results from certain patients presented in Example 2 are also provided. All patients were bevacizumab naïve.

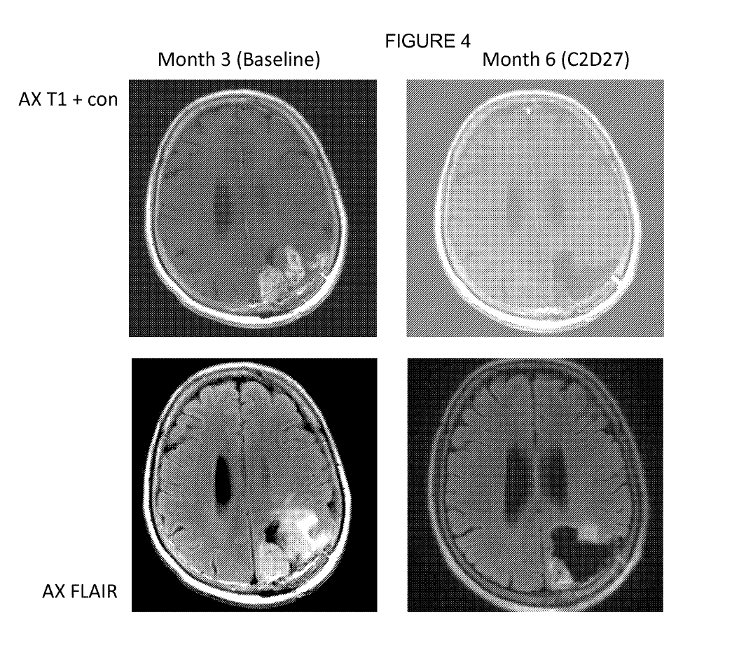

A first patient had a diagnosis of glioblastoma multiforme (MGMT−) in what is designated as month 1 with surgical resection in month 1, and adj temodar/RT during month 2. The patient was treated for 8 weeks with MEDI1/5 (1000 mg q2w) and bevacizumab (10 mg/kg q2w). No steroids were used through treatment. Brain scans at baseline (month 3) and after treatment (month 6) are provided in FIG. 4 showing a complete response (FLAIR showed a partial response).

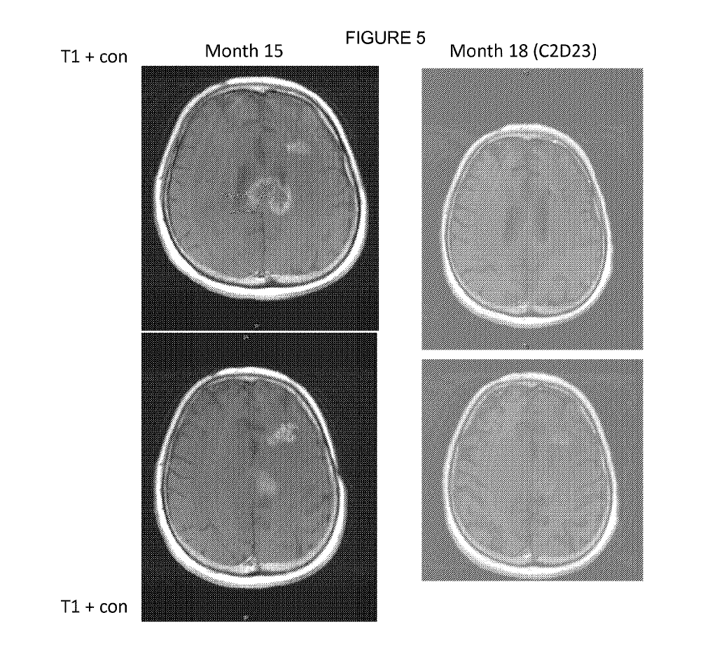

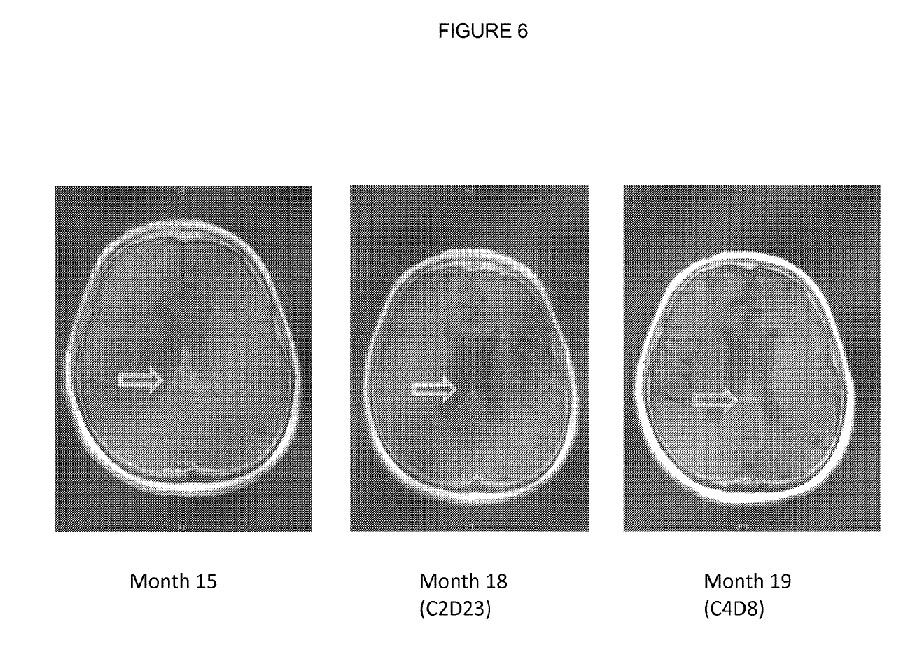

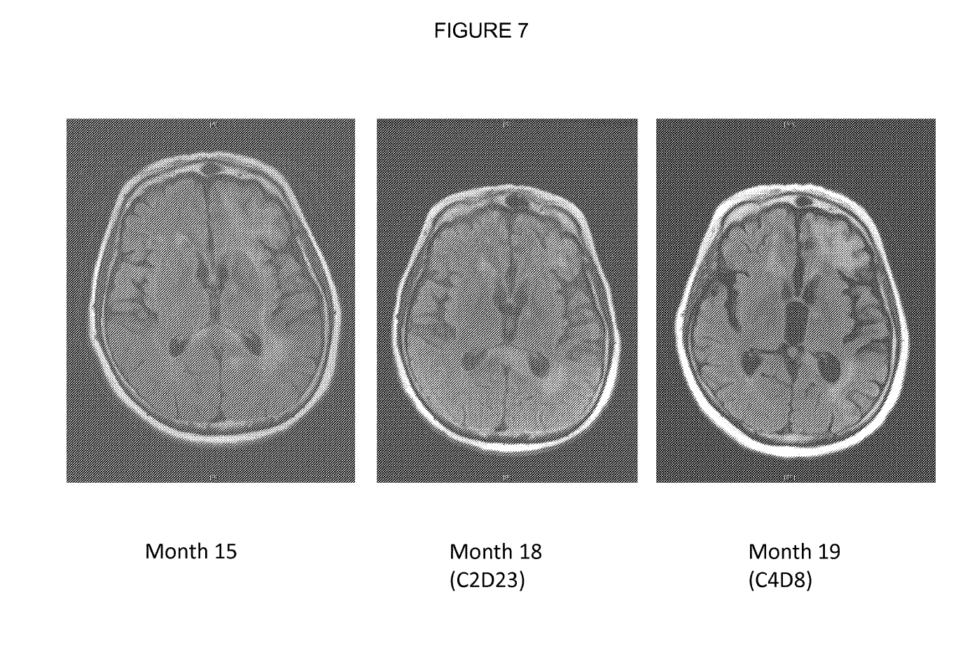

A second patient had a diagnosis of gliosarcoma (MGMT+) in what is designated as month 1. She received RT/temodar in month 14-15 with maintenance temodar ending on month 10. The patient was treated for 8 weeks with MEDI1/5 (1000 mg q2w) and bevacizumab (10 mg/kg q2w). A 24% reduction in the tumor was seen on the scans, as shown in FIG. 5 (T2 FLAIR also improved), see also FIG. 6 (C+ Axial), and FIG. 7 (Axial FLAIR).

A third patient had a diagnosis of glioblastoma multiforme (MGMT+) in what is designated month 1, with surgical resection at the same time, and adj temodar/RT from month 2 to month 13. The patient was treated for 8 weeks with MEDI1/5 (1000 mg q2w) and bevacizumab (10 mg/kg q2w). This patient did not demonstrate a response during the time period of the scan.

### Example 4

### Certain Embodiments

The following items provide certain embodiments disclosed herein.

1. A method of treating cancer in a patient comprising
a. providing an anti-Ang2 antibody or functional part thereof,
b. administering an anti-Ang2 antibody or functional part thereof to the patient, wherein the anti-Ang2 antibody or functional part thereof is administered at a dose from about 200 mg to about 1500 mg.

2. A method of inhibiting angiogenesis in a patient comprising
a. providing an anti-Ang2 antibody or functional part thereof,
b. administering an anti-Ang2 antibody or functional part thereof to the patient, wherein the anti-Ang2 antibody or functional part thereof is administered at a dose from about 200 mg to about 1500 mg.

3. The method of item 2, wherein the patient has cancer.

4. The method of any one of items 1-3, wherein the antibody or functional part thereof comprises the same heavy and light chain CDRs as MEDI1/5.

5. The method of any one of items 1-3, wherein the antibody or functional part thereof comprises a variable light chain comprising MEDI1; MEDI2; MEDI3; MEDI4; and MEDI6.

6. The method of any one of items 1-5, wherein the antibody or functional part thereof is an IgG1 or an IgG2 isotype antibody or functional part thereof.

7. The method of any one of items 1-6, wherein the antibody or functional part thereof further comprises a variable heavy chain region comprising MEDI5.

8. The method of any one of items 1-7, wherein the antibody or functional part thereof binds to the same epitope as any one of fully human monoclonal antibodies chosen from 3.19.3, MEDI1/5, MEDI2/5, MEDI3/5, MEDI6/5, and MEDI4/5.

9. The method of any one of items 1-7, wherein the antibody is a fully human monoclonal antibody chosen from: 3.19.3, MEDI1/5, MEDI2/5, MEDI3/5, MEDI6/5, and MEDI4/5.

10. The method of any one of items 1-7, wherein the functional part thereof is a functional part of a fully human monoclonal antibody chosen from: 3.19.3, MEDI1/5, MEDI2/5, MEDI3/5, MEDI6/5, and MEDI4/5.

11. The method of item 4, wherein the antibody or functional part thereof binds to the same epitope as MEDI1/5.

12. The method of item 11, wherein the antibody is MEDI1/5.

13. The method of item 11, wherein the functional part thereof is a functional part of the antibody MEDI1/5.

14. The method of any one of items 1-13, wherein the anti-Ang2 antibody or functional part thereof is administered at a dose from about 200 mg to about 1000 mg.

15. The method of any one of items 1-13, wherein the anti-Ang2 antibody or functional part thereof is administered at a dose from about 300 mg to about 1500 mg.

16. The method of item 15, wherein the anti-Ang2 antibody or functional part thereof is administered at a dose from about 1000 mg to about 1500 mg.

17. The method of any one of items 1-13, wherein the anti-Ang2 antibody or functional part thereof is administered at a dose of about 200 mg.

18. The method of any one of items 1-13, wherein the anti-Ang2 antibody or functional part thereof is administered at a dose of about 300 mg.

19. The method of any one of items 1-13, wherein the anti-Ang2 antibody or functional part thereof is administered at a dose of about 600 mg.

20. The method of any one of items 1-13, wherein the anti-Ang2 antibody or functional part thereof is administered at a dose of about 750 mg.

21. The method of any one of items 1-13, wherein the anti-Ang2 antibody or functional part thereof is administered at a dose of about 1000 mg.

22. The method of any one of items 1-13, wherein the anti-Ang2 antibody or functional part thereof is administered at a dose of about 1250 mg.

23. The method of any one of items 1-13, wherein the anti-Ang2 antibody or functional part thereof is administered at a dose of about 1500 mg.

24. The method of any one of items 1-23, wherein the anti-Ang2 antibody or functional part thereof is administered an IV infusion over from about 60 to about 90 minutes.

25. The method of item 24, wherein the anti-Ang2 antibody or functional part thereof is administered as an IV infusion over about 60 minutes.

26. The method of item 24, wherein the anti-Ang2 antibody or functional part thereof is administered as an IV infusion over about 90 minutes.

27. The method of item 25, wherein the anti-Ang2 antibody or functional part thereof is administered as an IV infusion over about 60 minutes and the dosage is less than about 1000 mg.

28. The method of item 26, wherein the anti-Ang2 antibody or functional part thereof is administered as an IV infusion over about 90 minutes and the dosage is greater than or equal to about 1000 mg.

29. The method of any one of items 1-28, wherein the patient receives one dosage.

30. The method of any one of items 1-28, wherein the patient receives multiple doses.

31. The method of item 30, wherein the dosage cycle is about every 14 days

32. The method of item 30, wherein the dosage cycle is about every 21 days.

33. The method of item 30, wherein the dosage cycle is about every 28 days.

34. The method of any one of items 30-33, wherein there are at least 2 dosage cycles.

35. The method of any one of items 30-33, wherein there are at least 3 dosage cycles.

36. The method of any one of items 30-33, wherein there are at least 4 dosage cycles.

37. The method of any one of items 30-33, wherein there are at least 5 dosage cycles.

38. The method of any one of items 30-33, wherein there are at least 6 dosage cycles.

39. The method of any one of items 30-33, wherein there are at least 7 dosage cycles.

40. The method of any one of items 30-33, wherein there are at least 8 dosage cycles.

41. The method of any one of items 30-33, wherein there are at least 9 dosage cycles.

42. The method of any one of items 30-33, wherein there are at least 10 dosage cycles.

43. The method of any one of items 30-33, wherein there are at least 11 dosage cycles.

44. The method of any one of items 30-33, wherein there are at least 12 dosage cycles.

45. The method of item 44, wherein there are more than 12 dosage cycles.

46. The method of item 44, wherein there are from 12 to 18 cycles.

47. The method of item 44, wherein there are from 12 to 31 cycles.

48. The method of any one of items 1-47, wherein the anti-Ang2 is coadministered with at least one additional therapeutic agent.

49. The method of item 48, wherein the anti-Ang2 is coadministered with two or more additional therapeutic agents.

50. The method of any one of items 48-49, wherein the coadministration is concurrent administration or sequential administration.

51. The method of item 50, wherein the sequential administration occurs on the same day.

52. The method of item 50, wherein the sequential administration occurs on different days.

53. The method of item 52, wherein the sequential administration occurs on different dosage cycles.

54. The method of item 52, wherein the sequential administration occurs on the same dosing cycle.

55. The method of any one of items 48-54, wherein at least one additional therapeutic agent is at least one chemotherapeutic agent.

56. The method of item 55, wherein the chemotherapeutic agent is chosen from at least one of carboplatin, capecitabine, gemcitabine, or paclitaxel.

57. The method of item 54, wherein the chemotherapeutic agent is carboplatin and paclitaxel.

58. The method of any one of items 55-57, wherein the chemotherapeutic agent is administered every week, every two weeks, every three weeks, or monthly.

59. The method of any one of items 56-57, wherein the paclitaxel is administered at about 175 mg/m².

60. The method of item 56, wherein the gemcitabine is administered at about 1000 mg/m².

61. The method of any one of items 56-57, wherein the carboplatin is administered at about AUC 4 or 5, wherein

total dose (mg)=(target AUC)×(GFR+25)

and wherein the GFR is estimated by using the serum creatinine level.

62. The method of item 61, wherein the mg dosage for an AUC of 5 is about 750 mg or less.

63. The method of item 61, wherein the mg dosage for an AUC of 4 is about 600 mg or less.

64. The method of any one of items 48-54, wherein the at least one additional therapeutic agent is cediranib.

65. The method of any one of items 48-5 4, wherein at least one additional therapeutic agent is at least one antibody or functional part thereof.

66. The method of item 65, wherein the antibody or functional part thereof is chosen from an anti-VEGF antibody or functional part thereof.

67. The method of item 66, wherein the antibody is bevacizumab.

68. The method of item 67, wherein the bevacizumab is administered at about 10 mg/kg or about 15 mg/kg.

69. The method of any one of items 67-68, wherein the bevacizumab is administered every two weeks or every three weeks.

70. The method of any one of items 1-69, wherein the patient has a Karnofsky performance status greater than or equal to about 60.

71. The method of item 70, wherein the patient has a Karnofsky performance status greater than or equal to about 70.

72. The method of any one of items 1-71, wherein the patient has breast cancer, colon cancer, lung cancer, renal cancer, prostate cancer, ovarian cancer, cervical cancer, liver cancer, gastric cancer, bladder cancer, skin cancer, leukemia, or brain cancer.

73. The method of any one of items 1-72, wherein the patient has ovarian cancer.

73. The method of item 72 wherein the skin cancer is melanoma.

74. The method of item 72, wherein the brain cancer is glioma.

75. The method of item 72, wherein the brain cancer is glioblastoma multiforme.

76. The method of item 72, wherein the lung cancer is non-small cell lung cancer.

77. The method of any one of items 1-71, wherein the cancer is melanoma, colon, colorectal, lung, small cell lung carcinoma, non-small cell lung carcinoma, breast, rectum, stomach, glioma, prostate, ovary, testes, thyroid, blood, kidney, renal cell carcinoma, liver, hepatocellular carcinoma, pancreas, brain, neck, esophageal, gastroesophageal, laryngeal, glioblastoma, endometrial cancer, cervical, testicular, and central nervous system cancer.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiments may be practiced in many ways and the claims include any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited value) that one of ordinary skill in the art would consider equivalent to the recited value (e.g., having the same function or result). In some instances, the term about may include numerical values that are rounded to the nearest significant figure.

What is claimed is:

1. A method of treating cancer or inhibiting angiogenesis in a patient comprising
a. providing an anti-Ang2 antibody or functional part thereof,
b. administering an anti-Ang2 antibody or functional part thereof to the patient,
wherein the anti-Ang2 antibody or functional part thereof is administered at a dose from about 200 mg to about 1500 mg.

2. The method of claim 1, wherein the antibody or functional part thereof comprises the same heavy and light chain CDRs as MEDI1/5.

3. The method of claim 2, wherein the antibody or functional part thereof is MEDI1/5 or a functional part thereof.

4. The method of claim 3, wherein the anti-Ang2 antibody or functional part thereof is administered at a dose from about 200 mg to about 1000 mg.

5. The method of claim 3, wherein the anti-Ang2 antibody or functional part thereof is administered at a dose from about 300 mg to about 1500 mg.

6. The method of claim 5, wherein the anti-Ang2 antibody or functional part thereof is administered at a dose from about 1000 mg to about 1500 mg.

7. The method of claim 6, wherein the anti-Ang2 antibody or functional part thereof is administered at a dose of about 1000 mg.

8. The method of claim 6, wherein the anti-Ang2 antibody or functional part thereof is administered at a dose of about 1500 mg.

9. The method of claim 3, wherein the anti-Ang2 antibody or functional part thereof is administered an IV infusion over from about 60 to about 90 minutes.

10. The method of claim 3, wherein the patient receives multiple doses.

11. The method of claim 10, wherein the dosage cycle is about every 14 days

12. The method of claim 10, wherein the dosage cycle is about every 21 days.

13. The method of claim 3, wherein the anti-Ang2 is coadministered with at least one additional therapeutic agent.

14. The method of claim 13, wherein at least one additional therapeutic agent is chosen from at least one of carboplatin, capecitabine, gemcitabine, or paclitaxel.

15. The method of claim 14, wherein at least one additional therapeutic agent is carboplatin and paclitaxel.

16. The method of claim 13, wherein at least one additional therapeutic agent is cediranib.

17. The method of claim 13, wherein at least one additional therapeutic agent is an anti-VEGF antibody or functional part thereof.

18. The method of claim 17, wherein the antibody is bevacizumab.

19. The method of claim 3, wherein the patient has ovarian cancer.

20. The method of claim 3, wherein the patient has glioblastoma multiforme.


SEQUENCE LISTING (2 sequences)

SEQ ID NO: 1 - protein, 123 aa, Artificial Sequence
  Description of Artificial Sequence Synthetic polypeptide
QVQLVESGGGVVQPGRSLRLSCAASGFTFTNYGMHWVRQAPGKGLEWVAVISHDGNNKYYVDSVKGRFTISRDNSKNTLYLQMNSLRAEDTAVYYCAREGIDFWSGLNWFDPWGQGTLVTVSS

SEQ ID NO: 2 - protein, 108 aa, Artificial Sequence
  Description of Artificial Sequence Synthetic polypeptide
EIVLTQSPGTLSLSPGERATLSCRASQSITGSYLAWYQQKPGQAPRLLITGASSWATGIPDRFSGSGSGTDFTLTISRLEPEDFAVYYCQQYSSSPITFGQGTRLEIK